(19) United States
(12) Patent Application Publication
(10) Pub. No.: US 2015/0216486 A1
(43) Pub. Date: Aug. 6, 2015

(54) NUCLEAR MEDICAL IMAGING APPARATUS AND CONTROLLING METHOD

(71) Applicants: Kabushiki Kaisha Toshiba, Minato-ku (JP); Toshiba Medical Systems Corporation, Otawara-shi (JP)

(72) Inventor: Kenta MORIYASU, Nasushiobara (JP)

(73) Assignees: Kabushiki Kaisha Toshiba, Minato-ku (JP); Toshiba Medical Systems Corporation, Otawara-shi (JP)

(21) Appl. No.: 14/609,941

(22) Filed: Jan. 30, 2015

(30) Foreign Application Priority Data
Jan. 31, 2014 (JP) 2014-017867

(51) Int. Cl. A61B 6/03; A61B 6/00

(52) U.S. Cl. CPC A61B 6/037; A61B 6/548; A61B 6/541

(57) ABSTRACT

A nuclear medical imaging apparatus according to an embodiment includes a driving unit and a controlling unit. The driving unit moves each of a plurality of image taking sites of a subject into an image taking region. When an image taking process for the subject is performed at each of the plurality of image taking sites, if data that has already been acquired from one of the image taking sites currently being imaged is determined to satisfy a predetermined condition, the controlling unit changes an image taking condition of the image taking process that is performed after the determination.

### CROSS-REFERENCE TO RELATED APPLICATIONS

This application is based upon and claims the benefit of priority from Japanese Patent Application No. 2014-017867, filed on Jan. 31, 2014, the entire contents of all of which are incorporated herein by reference.

### FIELD

Embodiments described herein relate generally to a nuclear medical imaging apparatus and a controlling method.

### BACKGROUND

Positron Emission computed Tomography (PET) apparatuses are a type of nuclear medical imaging apparatus reconstructs a PET image, which is a nuclear medical image, by acquiring data related to pair annihilation events from a subject to whom a drug labeled with a positron emission nuclide is administered. A PET apparatus reconstructs a PET image indicating a distribution of tissues of the subject that have taken the drug therein, by utilizing the phenomenon in which, when a pair annihilation event occurs as a result of the bonding of positrons emitted from the drug with electrons, two photons (two gamma rays) are emitted in substantially opposite directions.

Conventionally, during a PET medical examination (hereinafter, “PET examination”), an image taking process is performed on the subject at each of a plurality of image taking sites, with the use of a step-and-shoot method. According to the step-and-shoot method, after acquiring data at one of the image taking sites, the PET apparatus, for example, moves the position of the couch so as to acquire data at the next image taking site.

In this situation, for example, the PET apparatus performs the data acquisition process over a period of two to three minutes per image taking site. For this reason, when an image taking process is performed at each of a plurality of image taking sites during a PET examination, it can take a long time to finish the examination, and the examination may have low efficiency in some situations. When Single Photon Emission computed Tomography (SPECT) apparatuses, which are another type of nuclear medical imaging apparatus, are used, the examination may also have low efficiency in some situations, for the same reason.

### BRIEF DESCRIPTION OF THE DRAWINGS

FIG. 1 is a drawing for explaining an exemplary configuration of a PET apparatus according to a first embodiment;

FIG. 2 and is a drawing for explaining an example of count information;

FIG. 3A is a first drawing for explaining a coincidence list;

FIG. 3B is a second drawing for explaining the coincidence list;

FIG. 4 is a drawing for explaining an example of an image taking plan;

FIG. 5 is a first drawing for explaining a controlling unit according to the first embodiment;

FIG. 6 is a second drawing for explaining the controlling unit according to the first embodiment;

FIG. 7 is a flowchart for explaining an example of a process performed by the PET apparatus according to the first embodiment;

FIG. 8 is a drawing for explaining a controlling unit according to a second embodiment;

FIG. 9 is a flowchart for explaining an example of a process performed by a PET apparatus according to the second embodiment;

FIG. 10 is a drawing for explaining a first modification example of the second embodiment;

FIG. 11A is a first drawing for explaining a second modification example of the second embodiment;

FIG. 11B is a second drawing for explaining the second modification example of the second embodiment;

FIG. 12 is a drawing for explaining a third embodiment;

FIG. 13 is a flowchart for explaining an example of a process performed by a PET apparatus according to the third embodiment;

FIG. 14 is a flowchart for explaining an example of a process performed by a PET apparatus according to a fourth embodiment; and

FIG. 15 is a drawing for explaining a fifth embodiment.

### DETAILED DESCRIPTION

A nuclear medical imaging apparatus according to an embodiment includes a driving unit and a controlling unit. The driving unit moves each of a plurality of image taking sites of a subject into an image taking region. When an image taking process for the subject is performed at each of the plurality of image taking sites, if data that has already been acquired from one of the image taking sites currently being imaged is determined to satisfy a predetermined condition, the controlling unit changes an image taking condition of the image taking process that is performed after the determination.

Exemplary embodiments of a nuclear medical imaging apparatus will be explained in detail below, with reference to the accompanying drawings. In the following sections, exemplary embodiments of a Positron Emission computed Tomography (PET) apparatus, which is an example of the nuclear medical imaging apparatus, will be explained. In the following sections, the Position Emission computed Tomography apparatus will simply be referred to as a PET apparatus. The PET apparatus counts pairs of gamma rays (pair annihilation gamma rays) emitted from tissues having taken therein a drug that is administered to the subject and is labeled with a positron emission nuclide (e.g., 18F, which is a radioisotope). After that, the PET apparatus reconstructs PET image data indicating a distribution of the tissues that have taken the drug therein, on the basis of count information of the pair annihilation events. The pair annihilation events serve as an example of decay events of a radioisotope, and are events that occur in association with the decay of positrons.

### First Embodiment

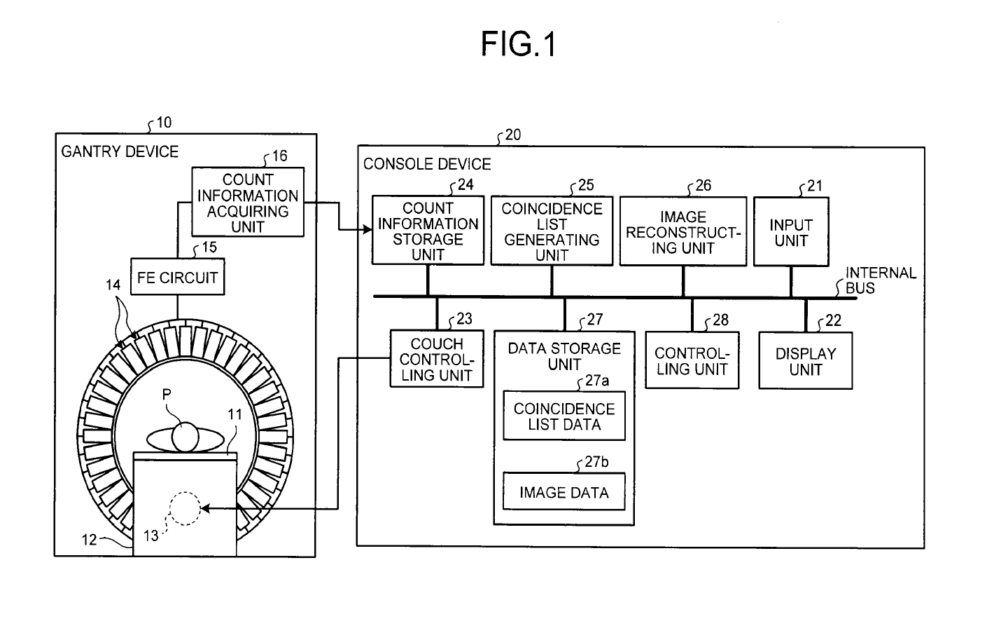

FIG. 1 is a drawing for explaining an exemplary configuration of a PET apparatus according to a first embodiment. As illustrated in FIG. 1, the PET apparatus according to the first embodiment includes a gantry device 10 and a console device 20.

The gantry device 10 is a device that counts, during a predetermined monitoring period (an image taking period), the gamma rays emitted by a positron emission nuclide that is administered to a subject P and is selectively taken into tissues in the body of the subject P. As illustrated in FIG. 1, the gantry device 10 includes a couchtop 11, a couch 12, a driving unit 13, detector modules 14, a Front End (FE) circuit 15, and a count information acquiring unit 16. As illustrated in FIG. 1, the gantry device 10 has a hollow serving as an image taking opening.

The couchtop 11 is a bed on which the subject P lies down and is positioned on top of the couch 12. Under control of a couch controlling unit 23 (explained later), the driving unit 13 moves an image taking site of the subject P into the space inside the image taking opening of the gantry device 10, by moving the couch 12. More specifically, the driving unit 13 moves the image taking site of the subject P into an image taking region that is provided inside the image taking opening and is called a “Field of View (FOV)”. The driving unit 13 is a moving mechanism used for moving, into the FOV, the image taking site from which data related to pair annihilation events, which serve as an example of decay events, are acquired. In the first embodiment, to move the image taking site of the subject P, the driving unit 13 may be configured to move the couchtop 11 or may be configured to move a gantry on which a detector (explained later) is installed.

The detector modules 14 are photon counting type detector that detects the gamma rays emitted from the subject P. For example, as illustrated in FIG. 1, the gantry device 10 according to the first embodiment is provided with the detector that includes the plurality of detector modules 14 disposed so as to surround the subject P in the form of a ring.

In an example, the detector modules 14 are each an Anger-type detector module and include scintillators, Photomultiplier Tubes (PMTs), and light guides. The scintillators are made of crystal of NaI, LYSO, BGO, or the like and are each converts gamma rays that are emitted from the subject P and become incident thereto, into visible light. In the detector modules 14, the plurality of scintillators are arranged two-dimensionally. Further, the PMTs are devices that multiply the visible light output from the scintillators and to convert the multiplied visible light into electric signals. The plurality of PMTs are densely arranged, while the light guides are interposed therebetween. The light guides are used for transferring the visible light output from the scintillators to the PMTs and are formed by using, for example, a plastic material or the like that has excellent light transmitting properties such as methyl methacrylate (MMA).

Each of the PMTs includes: a photocathode that receives scintillation light and generate photoelectrons; dynodes that are provided at multiple stages and that apply an electric field so as to accelerate the generated photoelectrons; and an anode that serves as an outflow port for electrons. The electrons emitted from the photocathode due to a photoelectric effect are accelerated toward a dynode and collide with the surface of the dynode, so as to knock out additional electrons. When this phenomenon is repeated at the multiple stages of dynodes, the number of electrons is multiplied in the manner of an avalanche so that the number of electrons reaches as many as approximately 1 million at the anode. In this example, the gain factor of the PMT is 1 million times. To cause this multiplication utilizing the avalanche phenomenon, a voltage of 600 volts or higher is usually applied to between the dynodes and the anode.

In other words, the detector modules 14 convert the gamma rays into the visible light with the use of the scintillators and further convert the converted visible light into the electric signals with the use of the PMTs.

The FE circuit 15 is connected at the subsequent stage (to the rear ends) of the plurality of PMTs included in each of the plurality of detector modules 14 and is connected at the previous stage (to the front end) of the count information acquiring unit 16. The FE circuit 15 outputs detection positions, energy levels, and detection times of the gamma rays, as count information of the detector including the plurality of detector modules 14. For example, on the basis of the electric signals output from the PMTs, the FE circuit 15 generates measurement data indicating the detection positions, the energy levels, and the detection times of the gamma rays by performing a measuring process described below and further outputs the generated measurement data to the count information acquiring unit 16 as the count information.

The FE circuit 15 measures the energy levels of the detected (counted) gamma rays by performing a waveform shaping process on analog waveform data of the electric signals output by the PMTs. For example, the FE circuit 15 generates data in which the wave height expresses energy levels, by performing calculating processes (an integral process and a differential process) on the analog waveform of the electric signals output by the PMTs. By using the generated data, the FE circuit 15 measures the energy levels (E) of the gamma rays converted into the visible light.

Further, on the basis of the analog waveform data of the electric signals output by the PMTs, the FE circuit 15 measures the times at which the gamma rays were detected (the detection times). For example, from the analog waveform data, the FE circuit 15 measures a point in time at which the voltage value has reached a predetermined threshold value as the detection time (T) of a gamma ray. In this situation, the detection time (T) may be expressed as an absolute time (the time) or as a relative time from an image taking starting time.

Further, the FE circuit 15 determines the incident positions of the gamma rays by performing, for example, an Anger-type position calculating process. More specifically, the FE circuit 15 calculates the positions of gravity points on the basis of the positions of a plurality of PMTs that converted a plurality of visible light beams output from the scintillators into electrical signals and output the converted electrical signals substantially at the same time with one another, as well as energy levels of the gamma rays corresponding to the strengths of these electric signals. After that, on the oasis of the positions of the gravity points obtained as a calculation result, the FE circuit 15 determines scintillator numbers (P) indicating the positions of the scintillators to which the gamma rays became incident. When the PMTs are position-detecting-type PMTs, the measurement data of the detection positions is output from the PMTs.

After that, the FE circuit 15 outputs the measurement data generated from the measuring process described above to the count information acquiring unit 16, as the count information of the detector. For example, the FE circuit 15 outputs “‘P: scintillator numbers’; ‘E: energy levels’; and ‘T: detection times’” that is kept in correspondence with a “module ID” used for uniquely identifying each of the detector modules 14, as the count information, to the count information acquiring unit 16.

The count information acquiring unit 16 acquires data related to decay events of a radioisotope. The count information acquiring unit 16 included in the PET apparatus illustrated in FIG. 1 acquires the data related to the pair annihilation events, as the data related to the decay events. More specifically, as the data related to the pair annihilation events, the count information acquiring unit 16 acquires the measurement information output by the FE circuit 15 and transmits the acquired count information to the console device 20. The count information acquiring unit 16 may also be referred to as an “acquiring unit”.

The console device 20 is a device that receives an operation performed on the PET apparatus by an operator and to reconstruct PET image data from the count information acquired by the gantry device 10. As illustrated in FIG. 1, the console device 20 includes an input unit 21, a display unit 22, the couch controlling unit 23, a count information storage unit 24, a coincidence list generating unit 25, an image reconstructing unit 26, a data storage unit 27, and a controlling unit 28. The units included in the console device 20 are connected to one another via an internal bus.

The input unit 21 includes a mouse, a keyboard, and/or the like used by the operator of the PET apparatus for inputting various types of instructions and various types of settings. The input unit 21 transfers information about the instructions and the settings received from the operator to the controlling unit 28. For example, the input unit 21 receives information related to an image taking plan for the subject P from the operator.

The display unit 22 is a monitor referred to by the operator and, under control of the controlling unit 28, displays image data and Graphical User Interface (GUI) used for receiving the various types of instructions and the various types of settings from the operator via the input unit 21.

The couch controlling unit 23 is a moving controlling unit that moves the image taking site of the subject P into the FOV provided on the inside of the image taking opening of the gantry device 10, by controlling the driving unit 13. Further, if a plurality of image taking sites are set in the image taking plan, the couch controlling unit 23 moves the subject P in such a manner that, for example, the center of each of the image taking sites in the body-axis direction is positioned at the center of the FOV provided on the inside of the image taking opening of the gantry device 10, by controlling the driving unit 13. In other words, the driving unit 13 moves each of the plurality of image taking sites of the subject P into the image taking region (FOV). When the driving unit 13 is a moving mechanism for the couchtop 11, the couch controlling unit 23 functions as a moving controlling unit constructed to control the moving of the couchtop 11. In contrast, when the driving unit 13 is a moving mechanism for the detector, the couch controlling unit 23 functions as a moving controlling unit constructed to control the moving of the gantry on which the detector is installed.

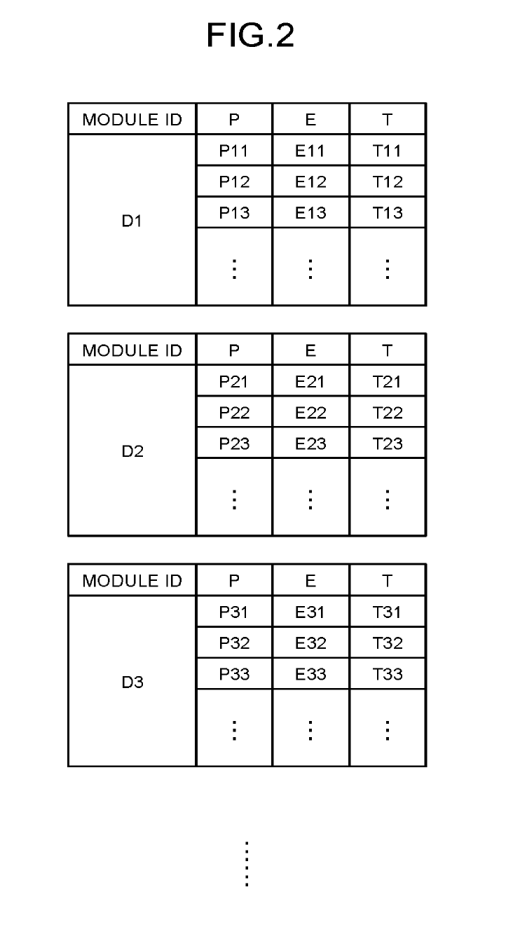

The count information storage unit 24 stores therein the count information acquired by the count information acquiring unit 16. FIG. 2 is a drawing for explaining an example of the count information.

For example, as illustrated in FIG. 2, the count information storage unit 24 stores therein “P: P11, E: E11, T: T11”, “P: P12, E: E12, T: T12”, and the like, as the count information acquired from the counted results by the detector module 14 identified with a “module ID: D1”. In FIG. 2, “P”, “E”, and “T” denote the “scintillator number”, the “energy level”, and the “detection time”, respectively.

Further, as illustrated in FIG. 2, the count information storage unit 24 similarly stores therein the count information acquired from the counted results by the detector modules 14 identified with a “module ID: D2” and a “module ID: D3”.

Returning to the description of FIG. 1, the coincidence list generating unit 25 searches the count information stored in the count information storage unit 24 for a plurality of pieces of count information in which a plurality of gamma rays emitted due to pair annihilation events are counted substantially at the same time. After that, the coincidence list generating unit 25 generates sets each made up of the plurality of pieces of count information found in the search, as a coincidence list (coincidence information).

Here, a PET examination performed by a PET apparatus will be explained further in detail. For a PET examination, a drug labeled with a positron emission nuclide is administered to the subject P. For example, to perform a PET examination for a cancer examination, 18F-labeled deoxyglucose labeled with “18F (fluorine)”, which is a positron emission nuclide, is administered to the subject P. The administered drug gathers at a specific site such as a tumor site of the subject P and emits positrons. A pair annihilation event occurs as a result of the bonding of the positrons with electrons in the surroundings thereof. Two photons (a pair of gamma rays) are emitted in a pair annihilation event. Because the total energy generated by the pair annihilation is “1022 keV”, the total energy of the two photons emitted due to the pair annihilation is “1022 keV”. Further, because of the law of conservation of momentum, the sum of the motion vectors of the two photons is a zero vector.

By utilizing the phenomenon in which, due to the pair annihilation event, the two photons (the two gamma rays) each having energy of 511 keV are emitted in substantially opposite directions, the PET apparatus reconstructs PET image data indicating a distribution of tissues of the subject that have taken the drug therein. Because of the law of conservation of momentum, the angle between the motion vectors of the two photons is substantially 180 degrees.

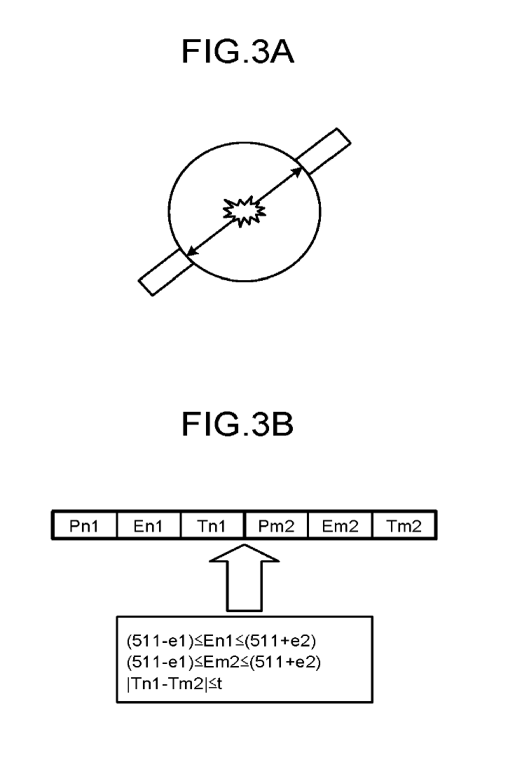

FIGS. 3A and 3B are drawings for explaining the coincidence list. As illustrated in FIG. 3A, the PET apparatus reconstructs the PET image data on the assumption that the location where a pair annihilation event occurred is positioned on a line connecting together the two detection positions (the scintillator numbers) in which the two photons were detected substantially simultaneously. The line connecting the two detection positions together is called a Line of Response (LOR).

For example, according to a condition (a coincidence list generating condition) that is set by the operator prior to the image taking process or set as an initial setting, the coincidence list generating unit 25 searches for two pieces of count information in which two photons were counted substantially simultaneously. After that, the coincidence list generating unit 25 generates the set made up of the two pieces of count information found in the search, as a coincidence list (coincidence information). In one example, as a coincidence list generating condition, a time window width “t” is set. In addition, as another coincidence list generating condition, an energy window width “(511−e1)≦E≦(511+e2)” may further be set, in some situations. In this situation, the values of “t”, “e1”, and “e2” are set, for example, in accordance with levels of precision in the measuring processes performed by the FE circuit 15 to measure the time and the energy.

Further, as illustrated in FIG. 3B, for example, the coincidence list generating unit 25 searches for two pieces of count information that satisfy “‘(511−e1)≦En1≦(511+e2)’; and ‘(511−e1)≦Em2≦(511+e2)’”. As a result, as illustrated in FIG. 3B, the coincidence list generating unit 25 generates a set made up of a piece of count information “P: Pn1; E: En1, T: Tn1” derived from the detector module 14 identified with the “module ID: n” and another piece of count information “P: Pm2; E: Em2, T: Tm2” derived from the detector module 14 identified with the “module ID: m”, as a coincidence list indicating the two photons that were detected substantially simultaneously.

After that, the coincidence list generating unit 25 stores the coincidence list into coincidence list data 27a stored in the data storage unit 27 illustrated in FIG. 1. The coincidence list represents projection data of the gamma rays (a sinogram).

Returning to the description of FIG. 1, the image reconstructing unit 26 reconstructs image data on the basis of the count information of the decay events. The image reconstructing unit 26 illustrated in FIG. 1 reconstructs image data (PET image data) on the basis of the count information of the pair annihilation events. In other words, the image reconstructing unit 26 reconstructs the PET image data by using the coincidence list stored in the coincidence list data 27a.

More specifically, the image reconstructing unit 26 reconstruct the PET image data by implementing a successive approximation method that uses the coincidence list. The image reconstructing unit 26 reconstructs PET image data for each of a plurality of axial planes, per image taking site. For example, the image reconstructing unit 26 reconstructs the PET image data by implementing a Maximum Likelihood Expectation Maximization (MLEM) method or an Ordered Subset MLEM (OSEM) method, as the successive approximation method. Alternatively, the image reconstructing unit 26 may perform the reconstructing process by using a time difference between detection times (a difference between times of flight) in the coincidence list, such as that implemented by a Time Of Flight (TOF)-PET apparatus.

Further, the image reconstructing unit 26 stores the PET image data into image data 27b included in the data storage unit 27 illustrated in FIG. 1. The first embodiment, however, is also applicable to a situation where the coincidence list is generated in the gantry device 10.

The controlling unit 28 exercises overall control of the PET apparatus, by controlling operations of the gantry device 10 and the console device 20. More specifically, the controlling unit 28 controls the moving of the couch 12 and controls the count information acquiring process performed by the count information acquiring unit 16. For example, the controlling unit 28 controls the moving of the image taking site by controlling the driving unit 13 via the couch controlling unit 23. Further, the controlling unit 28 controls the coincidence list generating process performed by the coincidence list generating unit 25 and the reconstructing process performed by the image reconstructing unit 26. Furthermore, the controlling unit 28 exercises control so that the image data stored in the image data 27b is displayed on the display unit 22.

An overall configuration of the PET apparatus according to the first embodiment has thus been explained. The PET apparatus according to the first embodiment as described above acquires, during the PET examination, the data related to the pair annihilation events of the subject P from each of the plurality of image taking sites and reconstructs the PET image data for each of the image taking sites. More specifically, the PET apparatus performs the image taking process on the subject P at each of the plurality of image taking sites, by implementing the step-and-shoot method. According to the step-and-shoot method, after acquiring data at one of the image taking sites, the PET apparatus moves the position of the subject P inside the gantry device 10, so as to acquire data at the next image taking site.

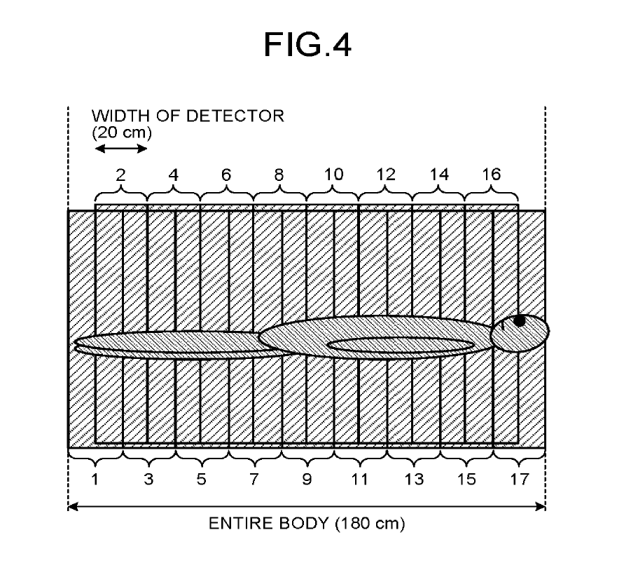

In this situation, the plurality of image taking sites are set, for example, as a result of an image taking plan for the PET image data made by an operator who viewed a scanogram of the subject P taken by an X-ray Computed Tomography (CT) apparatus. The scanogram is image data obtained by scanning the entire body of the subject P along the body axis direction, by moving the subject P along the body axis while X-rays are radiated thereon from an X-ray tube, while a rotation frame that rotatably supports the X-ray tube and an X-ray detector is fixed. FIG. 4 is a drawing for explaining an example of the image taking plan.

For instance, let us discuss an example in which the height of the subject P is “180 centimeters [cm]”, whereas the detector width (the width of the detector in the longitudinal direction of the couchtop 11) is “20 cm”. In that situation, for example, as illustrated in FIG. 4, the operator views the scanogram and configures a setting indicating that PET images of the entire body are taken in 17-time sessions, while the position of the subject P is moved so as to overlap by “10 cm” for every “20 cm”. In other words, the operator inputs the setting indicating that the PET images of image taking sites 1 to 17 are taken while the couch 12 is moved by 10 cm at a time. In the following description, each of the image taking sites may be referred to as a “bed position” or a “bed”.

In this situation, when an image taking process is performed at each of the plurality of image taking sites (by performing a multi-bed scan), for example, data is acquired over a period of two to three minutes per bed. To reconstruct images from the data acquired in the image taking process (the scan) corresponding to one bed, the calculation for the reconstructing process takes approximately four to five minutes according to currently-available conventional techniques. In other words, because the time period required by the image reconstructing process (the reconstruction period) is longer than the scan period (the image taking period), the operator is able to view the image data of the bed position from which the data is currently being acquired, only after the scan in the current bed position has been finished, according to the currently-available techniques. Because the operator needs to confirm that the scan has successfully been performed by checking the images, it takes a long period of time to complete the medical examination, and the medical examination thus has low efficiency.

However, if the processing capability of the image reconstructing unit 26 is improved as computing devices are developed, the reconstruction period may become shorter than the scan period per bed. Further, if the image reconstructing unit 26 was configured with a plurality of reconstruction processing units, and if the plurality of reconstruction processing units performed the reconstructing process in parallel to one another, the reconstruction period might become shorter than the scan period per bed.

In those situations, while a scan in a certain bed position is being performed, the operator would be able to view the PET image data in the bed position. Further, when the PET image data displayed on the display unit 22 as a result of the reconstructing process performed during the scan guarantees a sufficient level of image quality, the operator would be azole to end the image taking process performed at the image taking site from which the data is currently acquired and to start the image taking process at the next image taking site, in order to improve the workflow of the PET examination. However, no PET apparatus having such functions is available at present.

To cope with the situation, the controlling unit 28 included in the PET apparatus according to the first embodiment illustrated in FIG. 1 exercises control as described below in order to improve the efficiency of the medical examination: When an image taking process for the subject P is performed at each of a plurality of image taking sites, if the data that has already been acquired from one of the image taking sites currently being imaged is determined to satisfy a predetermined condition, the controlling unit 28 changes the image taking condition of the image taking process that is performed after the determination. For example, the predetermined condition is a condition about the image quality of image data obtained from the already-acquired image data. In the following explanation, the predetermined condition may be referred to as an image quality condition. The controlling unit 28 according to the first embodiment controls the driving unit 13 in such a manner that, if the data that has already been acquired by the count information acquiring unit 16 from one of the image taking sites that is currently being imaged is determined to satisfy the predetermined condition, an image taking process is started at the next image taking site following the image taking site currently being imaged.

More specifically, the controlling unit 28 according to the first embodiment causes the image reconstructing unit 26 to reconstruct image data (PET image data) on the basis of “the count information of the pair annihilation events (the coincidence list)”, which is the count information of decay events that have already been acquired from the image taking site currently being imaged. After that, the controlling unit 28 causes the display unit 22 to display the reconstructed image data (the PET image data). Subsequently, the controlling unit 28 determines that the predetermined condition is satisfied, when an imaging ending instruction indicating that the image taking process at the image taking site should be ended is received via the input unit 21 from the operator who viewed the image data (the PET image data) displayed on the display unit 22. In this situation, the control process by the controlling unit 28 according to the first embodiment is performed on the premise that the reconstruction period of the image reconstructing unit 26 is shorter than the image taking period (the scan period) per bed.

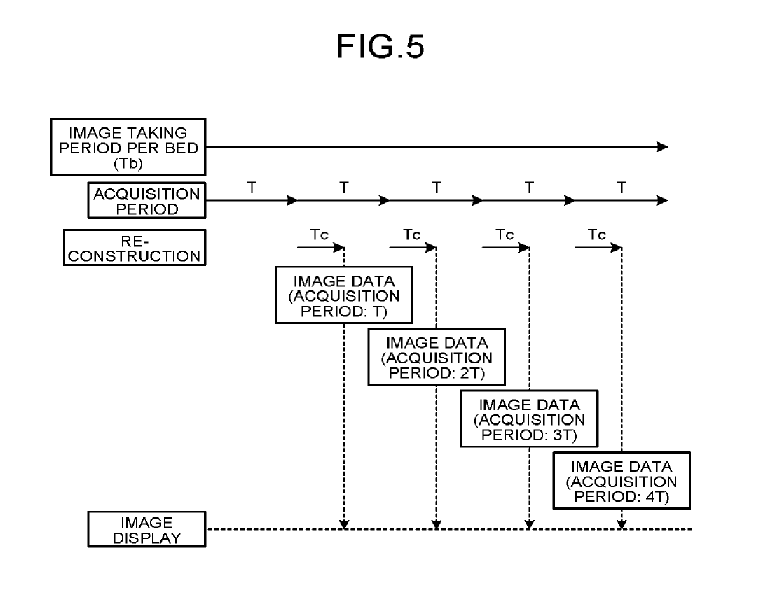

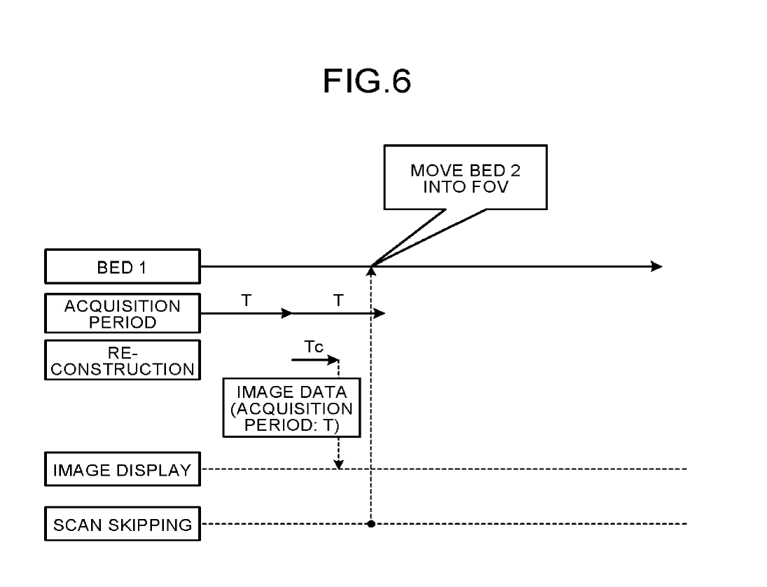

FIGS. 5 and 6 are drawings for explaining the controlling unit according to the first embodiment. FIG. 5 illustrates an example of information that is set for performing a scan skipping process according to the first embodiment. In FIG. 5, the image taking period (the scan period) per bed is expressed as “Tb”. Further, FIG. 5 illustrates an example in which each of the acquisition periods “T” is a time period equal to one fifth of the time period “Tb”. Further, FIG. 5 illustrates the example in which the reconstruction period “Tc” of the image reconstructing unit 26 satisfies “Tb>T>Tc”. In the first embodiment, because the operator is prompted to check the images a plurality of times for each bed position, it is desirable that “T>Tc” is satisfied. When the image reconstructing unit 26 is configured with reconstruction processing units of which the total quantity is “n”, where the reconstruction period of each of the reconstruction processing units is expressed as “Tu”, “T>Tc=Tu/n” is satisfied.

In that situation, as illustrated in FIG. 5, a reconstructing process is started when the time period “T” has elapsed since the start of an image taking process in a certain bed position. Image data is displayed at the point in time expressed as “T+Tc”, the image data being reconstructed from a coincidence list accumulated in the coincidence list data 27a by the end of the “acquisition period: T”. Further, as illustrated in FIG. 5, a reconstructing process is started when the time period “2T” has elapsed since the start of the image taking process, and image data is displayed at the point in time expressed as “2T+Tc”, the image data being reconstructed from a coincidence list accumulated in the coincidence list data 27a by the end of the “acquisition period: 2T”. Similarly, as illustrated in FIG. 5, image data is displayed at the point in time expressed as “3T+Tc”, the image data being reconstructed from a coincidence list accumulated in the coincidence list data 27a by the end of the “acquisition period: 3T”. Further, image data is displayed at the point in time expressed as “4T+Tc”, the image data being reconstructed from a coincidence list accumulated in the coincidence list data 27a by the end of the “acquisition period: 4T”.

The example of the setting illustrated in FIG. 5 is merely an example. “Tb” does not necessarily have to be an integer multiple of “T”. Further, the first embodiment may be applied to a situation where “T<Tc<Tb” is satisfied. Even in that situation, the image data is displayed at time intervals of “T”. Further, in the first embodiment, the image data for a scan skipping checking purpose may be is displayed once during the image taking process.

FIG. 6 is a drawing of a specific example of the scan skipping process performed with the exemplary setting illustrated in FIG. 5. For example, at the point in time expressed as “T+Tc” since the start of the image taking process at image taking site 1 (bed 1) illustrated in FIG. 4, the display unit 22 displays the image data reconstructed from the coincidence list of the “acquisition period: T”. After that, as illustrated in FIG. 6, if the operator has confirmed that the image data has a sufficient level of image quality and inputs a request for a “scan skipping process” via the input unit 21, the controlling unit 28 controls the driving unit 13 so as to move image taking site 2 (bed 2) into the image taking region (FOV). After that, the controlling unit 28 causes an image taking process to start at image taking site 2.

During the time period between when the image taking process at the currently-imaged image taking site is stopped and when the image taking process at the next image taking site is started, for example, the controlling unit 28 controls either the FE circuit 15 or the count information acquiring unit 16 so that the output of the count information is temporarily suspended, while the signals keep being output from the detector modules 14. Further, the controlling unit 28 controls either the FE circuit 15 or the count information acquiring unit 16 so that, for example, the output of the count information is resumed in response to the start of the image taking process at the next image taking site. In contrast, if no request for a scan skipping process is received from the operator, the controlling unit 28 continues the image taking process at bed 1.

As explained above, according to the first embodiment, during the scan in a certain bed position, an image reconstructing process is performed, for example, at the constant acquisition intervals (T) by using the data that has already been acquired. Every time an image reconstructing process is performed, the operator is prompted to view the result of the image reconstructing process so as to check the image quality. If the operator has confirmed that the image quality is at a sufficient level, the controlling unit 28 controls the driving unit 13 so that the scan in the current bed position is skipped and so that the next bed position of the subject P is moved into the image taking region.

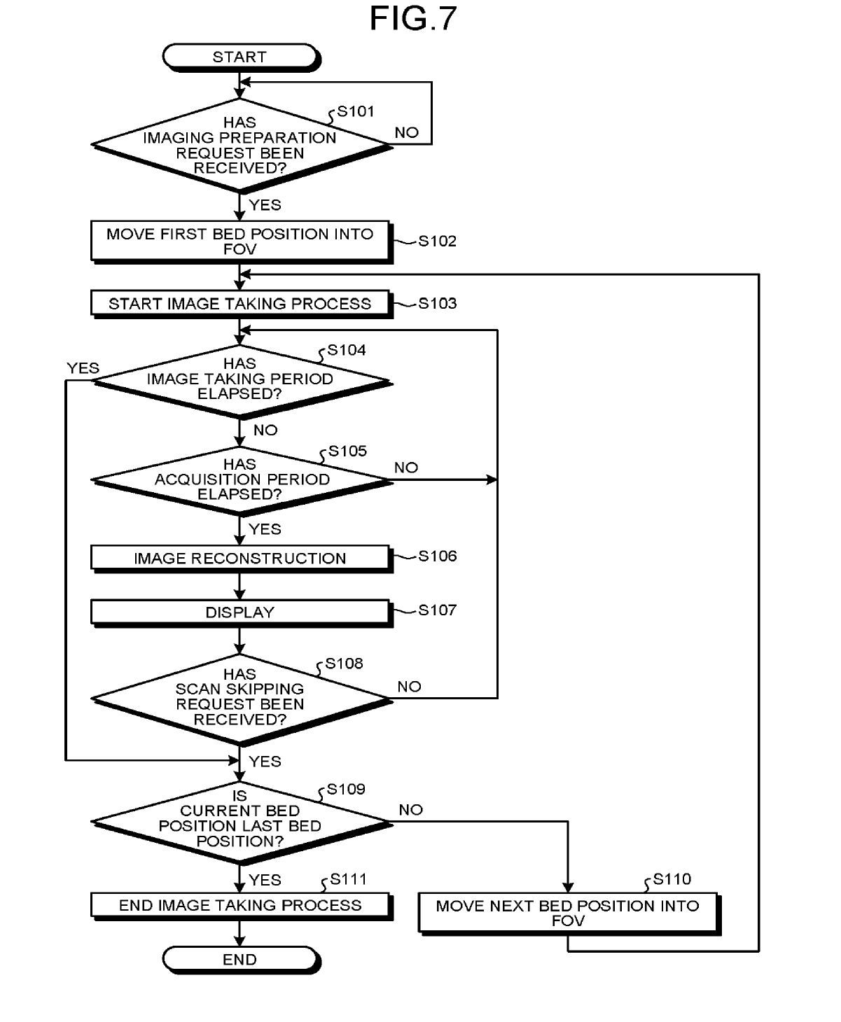

Next, a flow in a process performed by the PET apparatus according to the first embodiment will be explained, with reference to FIG. 7. FIG. 7 is a flowchart for explaining an example of the process performed by the PET apparatus according to the first embodiment.

As illustrated in FIG. 7, the controlling unit 28 included in the PET apparatus according to the first embodiment judges whether an imaging preparation request has been received from the operator via the input unit 21 (step S101). If no imaging preparation request has been received (step S101: No), the controlling unit 28 stands by until an imaging preparation request is received.

On the contrary, if an imaging preparation request has been received (step S101: Yes), the driving unit 13 moves the first bed position of the subject P into the FOV, under the control of the couch controlling unit 23 that received an instruction from the controlling unit 28 (step S102). After that, the couch controlling unit 23 notifies the controlling unit 28 that the moving has been completed. Subsequently, the controlling unit 28 instructs the various processing units to prepare to be able to perform an image taking process. After having received notifications from the various processing units that the preparation is completed, the controlling unit 28 causes the image taking process to start (step S103).

After that, the controlling unit 28 starts counting the time at the same time as the image taking process is started, and at first, judges whether the image taking period “Tb” has elapsed (step S104). If the image taking period “Tb” has not elapsed (step S104: No), the controlling unit 28 judges whether the acquisition period “T” has elapsed (step S105). If the acquisition period “T” has not elapsed (step S105: No), the process returns to step S104 where the controlling unit 28 judges whether the image taking period “Tb” has elapsed.

On the contrary, if the acquisition period “T” has elapsed (step S105: Yes), the image reconstructing unit 26 performs an image reconstructing process under the control of the controlling unit 28 (step S106), so that the display unit 22 displays the reconstructed image data (step S107). If the acquisition period “T” has elapsed, the controlling unit 28 resets the counted time and starts counting the time again.

After that, the controlling unit 28 judges whether a scan skipping request has been received from the operator via the input unit 21 (step S108). If no scan skipping request has been received (step S108: No), the controlling unit 28 continues the scan, and the process returns to step S104 where the controlling unit 28 judges whether the image taking period has elapsed.

On the contrary, if a scan skipping request has been received (step S108: Yes), the controlling unit 28 stops the image taking process and judges whether the current bed position is the last bed position (step S109). Also, if the image taking period “Tb” has elapsed without receiving any scan skipping request (step S104: Yes), the controlling unit 28 judges whether the current bed position is the last bed position (step S109). At the same time as the judging process at step S109, the controlling unit 28 stops counting the time for the acquisition period and initializes the count.

In this situation, if the current bed position is not the last bed position (step S109: No), the driving unit 13 moves the next bed position of the subject P into the FOV under the control of the controlling unit 28 (step S110), and the process returns to step S103 where the controlling unit 28 causes an image taking process to start at the post-move bed position.

On the contrary, if the current bed position is the last bed position (step S109: Yes), the controlling unit 28 ends the image taking process (step S111), and the process is thus ended. For example, the controlling unit 28 instructs the couch controlling unit 23 to lower the couchtop 11, so that the subject P can dismount from the couch 12.

If the image taking period “Tb” has elapsed without receiving any scan skipping request, the PET image data for the bed position is reconstructed from the coincidence list acquired during the image taking period “Tb”. Further, the image data that is reconstructed at step S106 and displayed at step S107 for the purpose of checking the image quality may be PET image data on a single axial cross-section in the bed position or may be a group of pieces of PET image data on a plurality of axial cross-sections. Further, in the first embodiment, the PET image data in the bed position of which the scan was skipped may be reconstructed again for an image diagnosis purpose, after the image taking processes for all the image taking sites have been finished, or the like.

Further, in the first embodiment, the operator may input a scan continuation request after the process at step S107, so as to prevent the images from being displayed many times during the image taking period “Tb”.

As explained above, according to the first embodiment, during the image taking process in a certain bed position, the operator is prompted to view the image data reconstructed from the data that has already been acquired. After that, in the first embodiment, if the operator who viewed the image data has confirmed that the image quality is at a sufficient level, the scan in the current bed position is skipped, so that the apparatus proceeds to the scan in the next bed position. As a result, it is possible to shorten the time period required by the PET examination. Consequently, according to the first embodiment, it is possible to improve the efficiency of the medical examination.

### Second Embodiment

In the first embodiment, the example is explained in which the efficiency of the medical examination is improved by having the operator check the image quality. In other words, in the first embodiment, the example is explained in which whether or not the operator has performed the input operation on the basis of his/her subjective judgment is used as the criterion for judging whether the image quality is satisfied. In contrast, in a second embodiment, an example will be explained in which the controlling unit 28 automatically performs a scan skipping process, by using a condition that can objectively be judged as the image quality condition.

A PET apparatus according to the second embodiment is configured to be similar to the PET apparatus according to the first embodiment explained with reference to FIG. 1. The controlling unit 28 according to the second embodiment, however, exercises control on the basis of the fact that the signal-noise (S/N) ratio, which is a parameter of the image quality, has a correlation with the quantity of pair annihilation events, which are decay events. For example, the S/N ratio is substantially proportional to the square root of the quantity of coincidence list entries. Further, the S/N ratio is positively correlated with the quantity of pieces of count information.

For this reason, the controlling unit 28 according to the second embodiment determines that the predetermined condition (the image quality condition) is satisfied, when the count of the pair annihilation events that have already been acquired from one of the image taking sites currently being imaged (the count of the decay events that have already been acquired) exceeds a predetermined threshold value. In the following sections, an example will be explained in which the controlling unit 28 uses the quantity of coincidence list entries stored in the coincidence list data 27a as the count of the pair annihilation events and performs a process by using a threshold value (Th) set for the quantity of coincidence list entries. Alternatively, in the second embodiment, the controlling unit 28 may use the quantity of pieces of count information stored in the count information storage unit 24 as the count of the pair annihilation events and perform a process by using a threshold value set for the quantity of pieces of count information.

The control process performed by the controlling unit 28 according to the second embodiment is also applicable to a situation where the reconstruction period of the image reconstructing unit 26 is longer than the image taking period (the scan period) per bed.

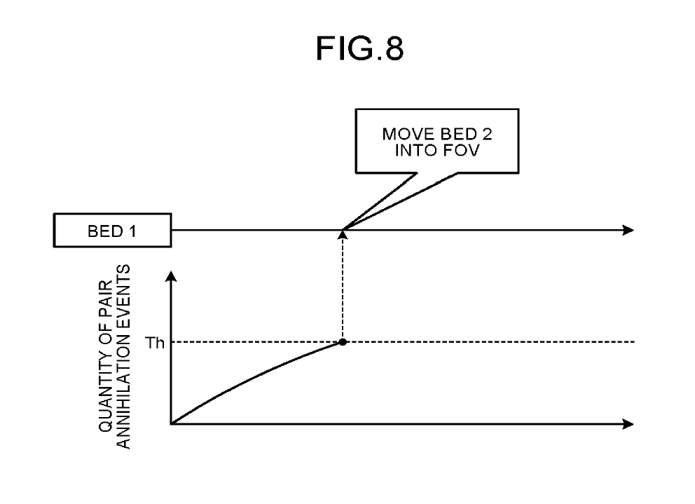

FIG. 8 is a drawing for explaining a controlling unit according to the second embodiment. For example, as illustrated in FIG. 8, the controlling unit 28 counts the quantity of pair annihilation events from the start of the image taking process at bed 1. After that, as illustrated in FIG. 8, when the quantity of pair annihilation events exceeds “Th”, the controlling unit 28 ends the image taking process at bed 1 and controls the driving unit 13 so as to move bed 2 into the image taking region (FOV). Subsequently, the controlling unit 28 causes an image taking process to start at image taking site 2.

In the second embodiment, the diagnosis purpose image reconstructing process at each of the image taking sites may be performed after the image taking processes at all the image taking sites have been finished or may be performed while the data is being acquired at the next image taking site.

Further, in the second embodiment, the coincidence list generating unit 25 may notify the controlling unit 28 of the quantity of coincidence list entries in the currently-imaged bed position. Alternatively, the second embodiment may be a case that, when the coincidence list generating unit 25 has determined that the image quality condition is satisfied, the coincidence list generating unit 25 notifies the controlling unit 28 that the image quality condition is satisfied. Further, the second embodiment may be a case that, when the quantity of pieces of count information is used as the quantity of pair annihilation events, the count information acquiring unit 16 notifies the controlling unit 28 of the counted value for the pieces of count information. Alternatively, the second embodiment may be a case that, when the count information acquiring unit 16 has determined that the image quality condition is satisfied, the count information acquiring unit 16 notifies the controlling unit 28 that the image quality condition is satisfied.

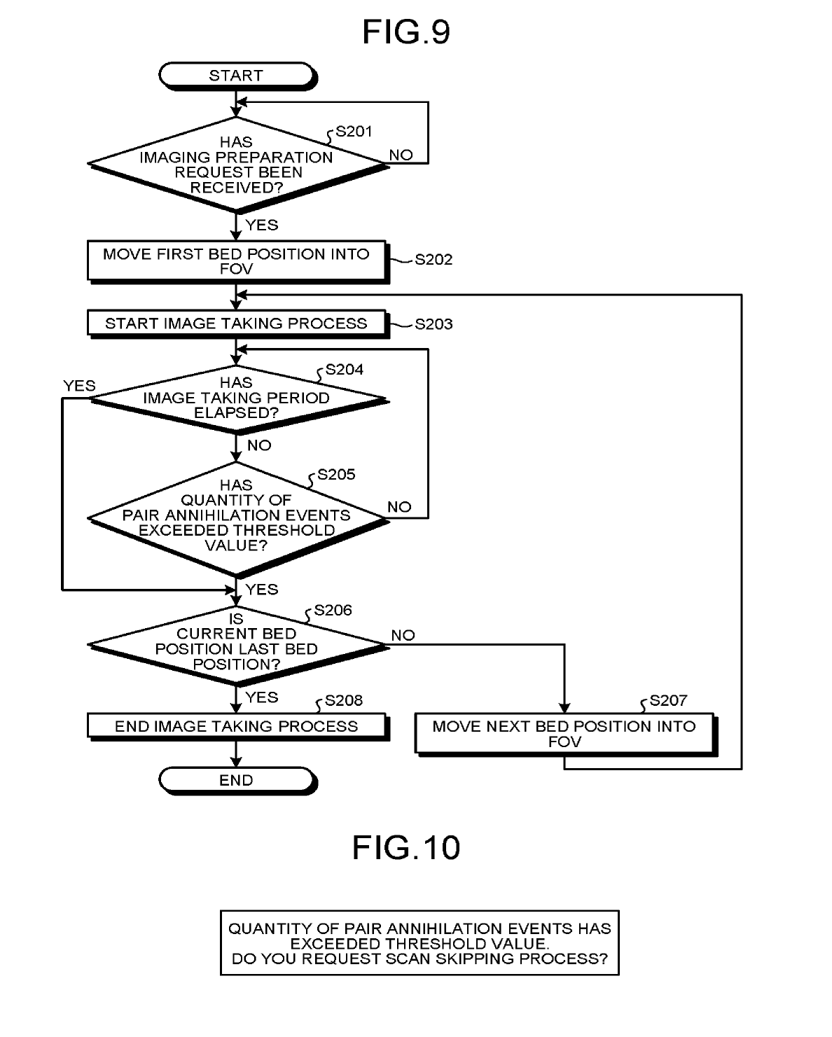

Next, a flow in a process performed by the PET apparatus according to the second embodiment will be explained, with reference to FIG. 9. FIG. 9 is a flowchart for explaining an example of the process performed by the PET apparatus according to the second embodiment.

As illustrated in FIG. 9, the controlling unit 28 included in the PET apparatus according to the second embodiment judges whether an imaging preparation request has been received from the operator via the input unit 21 (step S201). If no imaging preparation request has been received (step S201: No), the controlling unit 28 stands by until an imaging preparation request is received.

On the contrary, if an imaging preparation request has been received (step S201: Yes), the driving unit 13 moves the first bed position of the subject P into the FOV, under the control of the couch controlling unit 23 that received an instruction from the controlling unit 28 (step S202). After that, the couch controlling unit 23 notifies the controlling unit 28 that the moving has been completed. Subsequently, the controlling unit 28 instructs the various processing units to prepare to be able to perform an image taking process. After having received notifications from the various processing units that the preparation is completed, the controlling unit 28 causes the image taking process to start (step S203).

After that, the controlling unit 28 judges whether the image taking period “Tb” has elapsed (step S204). If the image taking period “Tb” has not elapsed (step S204: No), the controlling unit 28 judges whether the quantity of pair annihilation events has exceeded the threshold value (step S205). If the quantity of pair annihilation events has not exceeded the threshold value (step S205: No), the process returns to step S204 where the controlling unit 28 judges whether the image taking period “Tb” has elapsed.

On the contrary, if the quantity of pair annihilation events has exceeded the threshold value (step S205: Yes), the controlling unit 28 judges whether the current bed position is the last bed position (step S206). Also, if the image taking period “Tb” has elapsed without the quantity of pair annihilation events exceeding the threshold value (step S204: Yes), the controlling unit 28 judges whether the current bed position is the last bed position (step S206).

In this situation, if the current bed position is not the last bed position (step S206: No), the driving unit 13 moves the next bed position of the subject P into the FOV under the control of the controlling unit 28 (step S207), and the process returns to step S203 where the controlling unit 28 causes an image taking process to start at the post-move bed position.

On the contrary, if the current bed position is the last bed position (step S206: Yes), the controlling unit 28 ends the image taking process (step S208), and the process is thus ended. For example, the controlling unit 28 instructs the couch controlling unit 23 to lower the couchtop 11, so that the subject P can dismount from the couch 12.

As explained alcove, in the second embodiment, the quantity of pair annihilation events in the bed position currently being imaged is monitored. If a certain quantity of pair annihilation events that guarantees a sufficient level of image quality has been counted, the scan in the bed position is automatically skipped, so that the apparatus proceeds to the scan in the next bed position. As a result, according to the second embodiment, it is possible to shorten the time period required by the PET examination. In addition, it is possible to reduce the work of the operator during the image taking process and to further improve the efficiency of the medical examination.

When the control process according to the second embodiment is performed, it is acceptable to implement any of the three modification examples described below:

In a first modification example of the second embodiment, when the count of the pair annihilation events that have already been acquired from the image taking site currently being imaged exceeds the predetermined threshold value, the controlling unit 28 inquires the operator whether the image taking process at the image taking site should be ended. If an imaging ending instruction is received from the operator via the input unit 21, the controlling unit 28 determines that the predetermined condition (the image quality condition) is satisfied.

FIG. 10 is a drawing for explaining the first modification example of the second embodiment. For example, when the quantity of pair annihilation events has exceeded “Th”, as illustrated in FIG. 10, the controlling unit 28 causes the display unit 22 to display a message that reads “The quantity of pair annihilation events has exceeded the threshold value. Do you request a scan skipping process?”.

After that, for example, when the operator inputs a scan skipping request by using the input unit 21, the controlling unit 28 performs the scan skipping process. On the contrary, for example, if the operator inputs a scan continuation request by using the input unit 21, the controlling unit 28 does not perform the scan skipping process and continues the image taking process at the appropriate bed position.

In the first modification example described above, because the operator is notified that the quantity of pair annihilation events has exceeded the threshold value so that the operator is consigned to make the final judgment about the scan skipping process, it is possible to avoid the situation where the scan skipping process is automatically performed against the operator's intention.

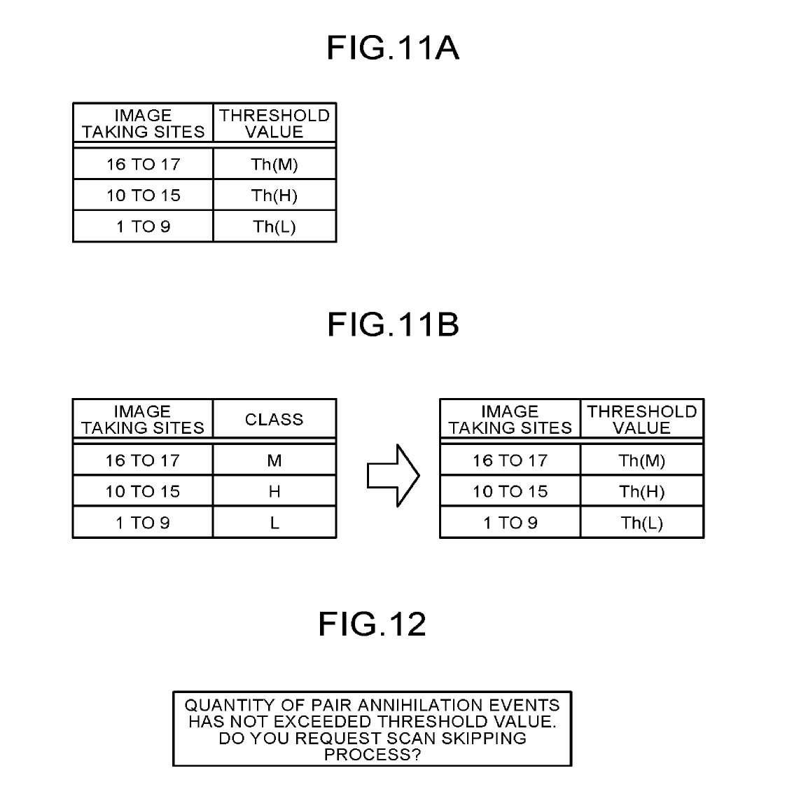

In a second modification example of the second embodiment, the controlling unit 28 judges whether the image quality condition is satisfied, by using a threshold value that is set for each of the plurality of image taking sites. FIGS. 11A and 115 are drawings for explaining the second modification example of the second embodiment.

For example, the operator sets a threshold for the quantity of coincidence list entries for each of the image taking sites, when making the image taking plan explained with reference to FIG. 4. For example, as illustrated in FIG. 11A, for the image taking sites (1 to 9) including the lower limbs, the upper limbs, and the lower abdomen, and the like, the operator sets a small threshold value “Th(L)”, because these image taking sites have lower importance in the image diagnosis process for the subject P. In addition, for example, as illustrated in FIG. 11A, for the image taking sites (10 to 15) including the abdomen, the chest, and the neck, and the like, the operator sets a large threshold value “Th(H)”, because these image taking sites have higher importance in the image diagnosis process for the subject P. Further, for example, as illustrated in FIG. 11A, for the image taking sites (16 and 17) including the head, the operator sets a threshold value “Th(M)”, which falls between Th(L) and Th(H), because these image taking sites have medium importance in the image diagnosis process for the subject P.

In another example, as illustrated on the left-hand side of FIG. 11B, the operator sets image taking sites 1 to 9 to a class with an importance level “L”, sets image taking sites 10 to 15 to a class with an importance level “H”, and sets image taking sites 16 and 17 to a class with an importance level “M”. In this situation, the controlling unit 28 stores therein settings in which the threshold value “Th(L)” is kept in correspondence with the “class: L”, the threshold value “Th(M)” is kept in correspondence with the “class: M”, and the threshold value “Th(H)” is kept in correspondence with the “class: H”.

In that situation, as illustrated cn the left-hand side of FIG. 11B, the controlling unit 28 automatically sets “Th(L)” for image taking sites 1 to 9, automatically sets “Th(H)” for image taking sites 10 to 15, and automatically sets “Th(M)” for image taking sites 16 and 17.

When the settings illustrated in FIG. 11A or 11B are made, to perform the judging process at step S205 in FIG. 9, the controlling unit 28 uses “Th(L)” for image taking sites 1 to 9, uses “Th(H)” for image taking sites 10 to 15, and uses “Th(M)” for image taking sites 16 and 17.

In the second modification example described above, because the threshold values used in the automatic judgment for the scan skipping process are set in accordance with the levels of image quality required by the image diagnosis process, it is possible to avoid, for example, the situation where a re-examination needs to be performed.

In a third modification example of the second embodiment, the controlling unit 28 judges whether the predetermined condition (the image quality condition) is satisfied, by using a threshold value that is set in accordance with at least one of body information and pathological information of the subject P. In this situation, the body information may be, for example, the height, the weight, and the like of the subject P. The pathological information may be, for example, past medical history and current medical history of the subject P. The pathological information can also serve as a specific example of the levels of importance explained in the second modification example.

The gamma rays generated on the inside of the subject P pass through tissues of the subject's body and enter the detector. In this situation, by interacting the gamma ray interacts with the tissues, an event that the gamma ray is not incident onto a detector which the gamma ray should be incident may be occurred. For this reason, for example, the larger is the thickness of the body of the subject P, the lower the count value becomes. Accordingly, on the basis of the body information of the subject P, if the operator determines that the subject P is of an obese type, for example, the operator sets a threshold value larger than a reference threshold value based on the S/N ratio. In another example, if a malignant tumor of lymph nodes is recorded in the past medical history or the current medical history of the subject P, the operator sets a threshold value that is larger than the reference threshold value based on the S/N ratio, in consideration of the possibility of systemic metastasis of the cancer. In yet another example, if the subject P is of an obese type on the basis of the body information, and the subject P has the possibility of having systemic metastasis of cancer on the basis of the pathological information, the operator sets a threshold value that is even larger than the threshold values described above.

In the third modification example of the second embodiment, the controlling unit 28 may judge whether the predetermined condition (the image quality condition) is satisfied, by using a threshold value that is set for each of the plurality of image taking sites in accordance with at least one of the body information and the pathological information of the subject P. For example, the operator may set the threshold value for each of the image taking sites of the subject P, in accordance with the size of the image taking site. Further, for example, if stomach cancer is recorded in the past medical history or the current medical history of the subject P, the operator may set a threshold value for an image taking site at the abdomen to be larger than threshold values for other image taking site.

In the third modification example described above, because the threshold values used in the automatic judgment for the scan skipping process are set in accordance with the levels of image quality required by the image diagnosis process for the subject P, it is possible to avoid, for example, the situation where a re-examination needs to be performed. As a modification example of the second embodiment, for instance, both the first and the second modification examples or both the first and the third modification examples may be implemented.

### Third Embodiment

In a third embodiment, an example will be explained in which when the control process described in the first embodiment is performed, the control process described in the second embodiment is also performed, with reference to FIG. 12 and the like. FIG. 12 is a drawing for explaining the third embodiment.

In the third embodiment, as explained in the first embodiment, the image data for the image quality checking purpose is displayed. After that, at the point in time when an imaging ending instruction (a scan skipping request) for the image taking site currently being imaged is received from the operator via the input unit 21, the controlling unit 28 according to the third embodiment judges whether the count of the pair annihilation events that have already been acquired (the count of the decay events that have already been acquired) has exceeded a threshold value or not. If the count of the pair annihilation events has not exceeded the threshold value, the controlling unit 28 according to the third embodiment inquires the operator whether the image taking process at the image taking site should be ended or not.

For example, as illustrated in FIG. 12, the controlling unit 28 causes the display unit 22 to display a message that reads “The quantity of pair annihilation events has not exceeded the threshold value. Do you request a scan skipping process?”.

After that, if an imaging ending instruction is received again (a scan skipping re-request) from the operator via the input unit 21, the controlling unit 28 according to the third embodiment determines that the predetermined condition (the image quality condition) is satisfied. In the third embodiment also, the controlling unit 28 may make an inquiry to the operator by using a threshold value that is set for each of the plurality of image taking sites, similarly to the second modification example of the second embodiment. Further, in the third embodiment also, the controlling unit 28 may make an inquiry to the operator, by using either a threshold value that is set in common to all the image taking sites in accordance with at least one of the body information and the pathological information of the subject P or a threshold value that is set for each of the plurality of image taking sites in accordance with at least one of the body information and the pathological information of the subject P, similarly to the third modification example of the second embodiment.

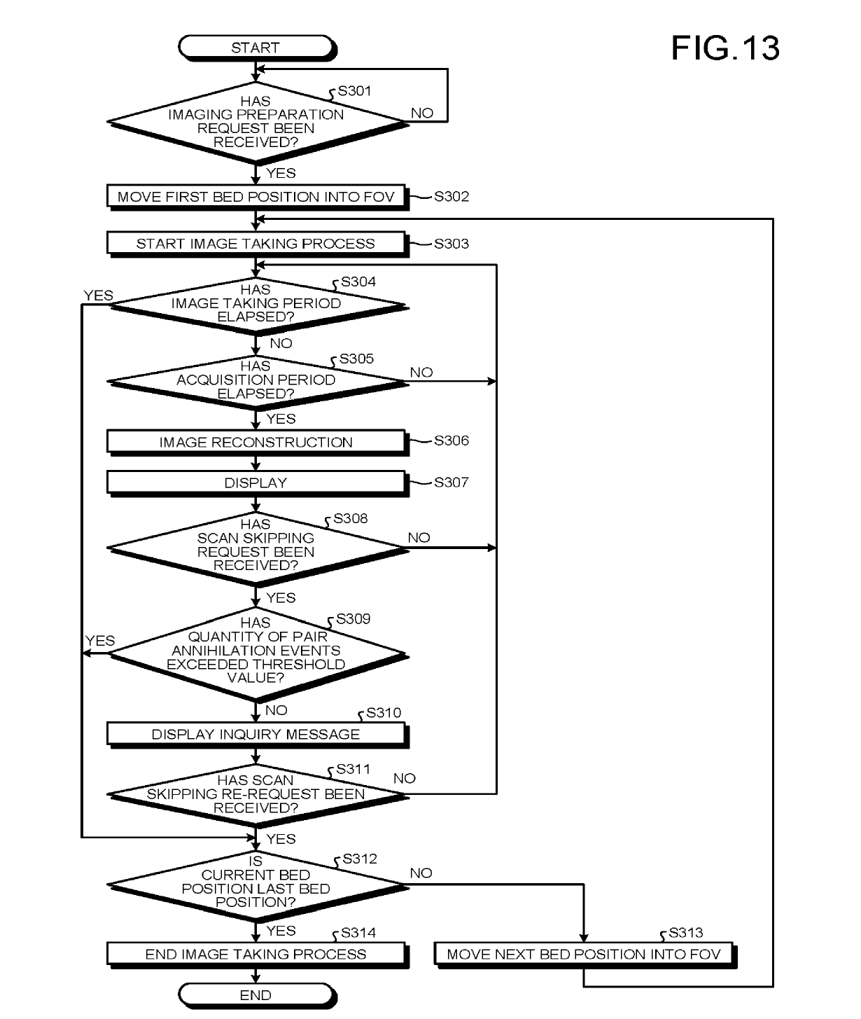

Next, a flow in a process performed by the PET apparatus according to the third embodiment will be explained, with reference to FIG. 13. FIG. 13 is a flowchart for explaining an example of the process performed by the PET apparatus according to the third embodiment.

As illustrated in FIG. 13, the controlling unit 28 included in the PET apparatus according to the third embodiment judges whether an imaging preparation request has been received from the operator via the input unit 21 (step S301). If no imaging preparation request has been received (step S301: No), the controlling unit 28 stands by until an imaging preparation request is received.

On the contrary, if an imaging preparation request has been received (step 3301: Yes), the driving unit 13 moves the first bed position of the subject P into the FOV, under the control of the couch controlling unit 23 that received an instruction from the controlling unit 28 (step S302). Subsequently, the controlling unit 28 causes the image taking process to start (step 3303).

After that, the controlling unit 28 starts counting the time at the same time as the image taking process is started and judges whether the image taking period “Tb” has elapsed (step S304). If the image taking period “Tb” has not elapsed (step S304: No), the controlling unit 28 judges whether the acquisition period “T” has elapsed (step S305). If the acquisition period “T” has not elapsed (step S305: No), the process returns to step S304 where the controlling unit 28 judges whether the image taking period “Tb” has elapsed.

On the contrary, if the acquisition period “T” has elapsed (step S305: Yes), the image reconstructing unit 26 performs an image reconstructing process under the control of the controlling unit 28 (step S306), so that the display unit 22 displays the reconstructed image data (step S307).

After that, the controlling unit 28 judges whether a scan skipping request has been received from the operator via the input unit 21 (step S308). If no scan skipping request has been received (step S308: No), the controlling unit 28 continues the scan, and the process returns to step S304 where the controlling unit 28 judges whether the image taking period has elapsed.

On the contrary, if a scan skipping request has been received (step S308: Yes), the controlling unit 28 judges whether the quantity of pair annihilation events has exceeded the threshold value (step S309). If the quantity has not exceeded the threshold value (step S309: No), the display unit 22 displays the inquiry message under the control of the controlling unit 28 (step S310). After that, the controlling unit 28 judges whether a scan skipping re-request has been received from the operator (step S311). If no scan skipping re-request has been received (step S311: No), the controlling unit 28 determines that the scan should be continued, and the process returns to step S304 where the controlling unit 28 judges whether the image taking period “Tb” has elapsed.

On the contrary, if the quantity of pair annihilation events has exceeded the threshold value (step S309: Yes) or if a scan skipping re-request has been received (step S311: Yes), the controlling unit 28 stops the image taking process and judges whether the current bed position is the last bed position (step S312). Also, if the image taking period “Tb” has elapsed without receiving any scan skipping request or any scan skipping re-request (step S304: Yes), the controlling unit 28 judges whether the current bed position is the last bed position (step S312).

In this situation, if the current bed position is not the last bed position (step S312: No), the driving unit 13 moves the next bed position of the subject P into the FOV under the control of the controlling unit 28 (step S313), and the process returns to step S303 where the controlling unit 28 causes an image taking process to start at the post-move bed position.

On the contrary, if the current bed position is the last bed position (step S312: Yes), the controlling unit 28 ends the image taking process (step S314), and the process is thus ended.

The description of the first embodiment and the description of the second embodiment are also applicable to the third embodiment, except that the inquiry is made to the operator on the basis of the quantity of pair annihilation events.

As explained above, according to the third embodiment, even if the operator who viewed the image data reconstructed from the data that has already been acquired that is currently being imaged has confirmed that a sufficient level of image quality is guaranteed on the basis of his/her subjective judgment, it is automatically judged whether the image quality of the image data is suitable for the diagnosis purpose on the basis of the objective criterion. Further, in the third embodiment, if there is a possibility that the image quality of the image data may not be suitable for the diagnosis purpose according to the objective criterion, the operator views the inquiry message and is able to judge again whether the scan skipping process should be performed. As a result, according to the third embodiment, for example, it is possible to reduce the possibility of having to perform the image taking process again and to thus improve the workflow of the PET examination.

### Fourth Embodiment

In a fourth embodiment, an example will be explained in which, when the control process described in the second embodiment is performed, the control process described in the first embodiment is also performed.

The controlling unit 28 according to the fourth embodiment compares the quantity of pair annihilation events, which is the quantity of decay events, with the threshold value, as explained in the second embodiment. Any of the threshold values described in the second embodiment, the second modification example of the second embodiment, and the third modification example of the second embodiment is usable as the threshold value in the fourth embodiment. Further, when the count of the pair annihilation events that have already been acquired (the count of the decay events that have already been acquired) from the image taking site currently being imaged has exceeded the threshold value, the controlling unit 28 according to the fourth embodiment causes the image reconstructing unit 26 to reconstruct image data (PET image data) on the basis of the counted result of the pair annihilation events that have already been acquired.

After that, the controlling unit 28 causes the display unit 22 to display the image data (the PET image data). Subsequently, if an imaging ending instruction (a scan skipping request) for the image taking site is received via the input unit 21 from the operator who viewed the image data (the PET image data) displayed on the display unit 22, the controlling unit 28 determines that the predetermined condition (the image quality condition) is satisfied and causes an image taking process to start at the next image taking site.

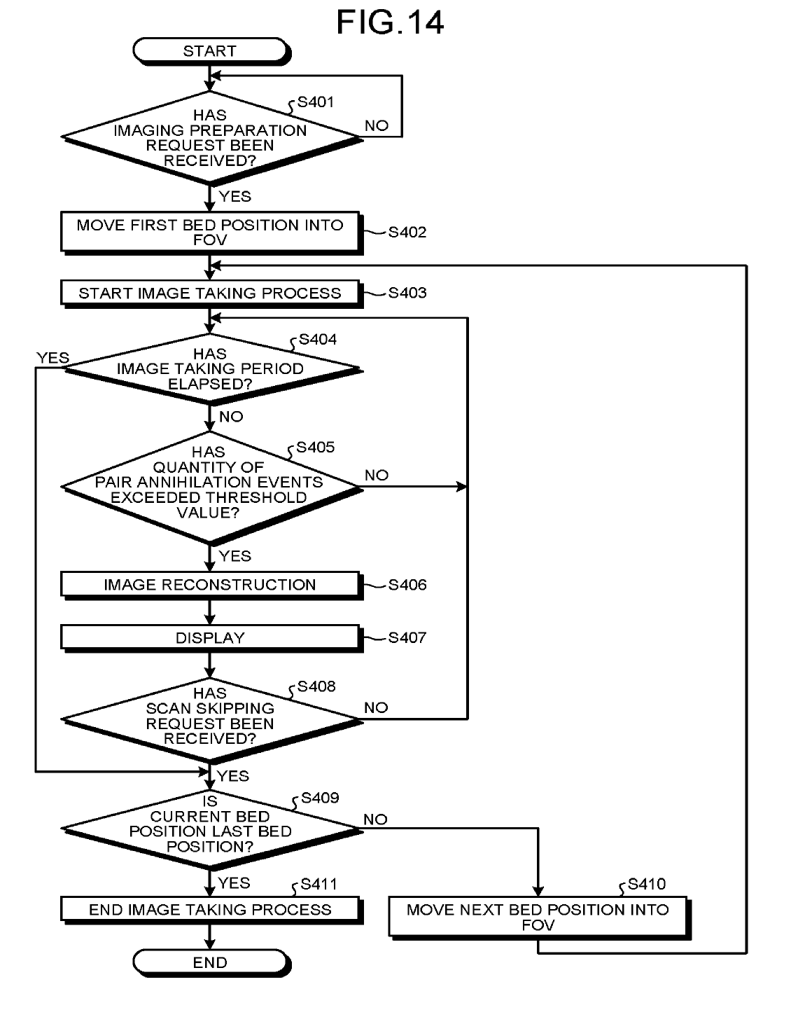

Next, a flow in a process performed by the PET apparatus according to the fourth embodiment will be explained, with reference to FIG. 14. FIG. 14 is a flowchart for explaining an example of the process performed by the PET apparatus according to the fourth embodiment.

As illustrated in FIG. 14, the controlling unit 28 included in the PET apparatus according to the fourth embodiment judges whether an imaging preparation request has been received from the operator via the input unit 21 (step S401). If no imaging preparation request has been received (step S401: No), the controlling unit 28 stands by until an imaging preparation request is received.

On the contrary, if an imaging preparation request has been received (step S401: Yes), the driving unit 13 moves the first bed position of the subject P into the FOV, under the control of the couch controlling unit 23 that received an instruction from the controlling unit 28 (step S402). Subsequently, the controlling unit 28 causes the image taking process to start (step S403).

After that, the controlling unit 28 judges whether the image taking period “Tb” has elapsed (step S404). If the image taking period “Tb” has not elapsed (step S404: No), the controlling unit 28 judges whether the quantity of pair annihilation events has exceeded the threshold value (step S405). If the quantity of pair annihilation events has not exceeded the threshold value (step S405: No), the process returns to step S404 where the controlling unit 28 judges whether the image taking period “Tb” has elapsed.

On the contrary, if the quantity of pair annihilation events has exceeded the threshold value (step S405: Yes), the image reconstructing unit 26 performs an image reconstructing process under the control of the controlling unit 28 (step S406), so that the display unit 22 displays the reconstructed image data (step S407).

After that, the controlling unit 28 judges whether a scan skipping request has been received from the operator via the input unit 21 (step S408). If no scan skipping request has been received (step S408: No), the controlling unit 28 continues the scan, and the process returns to step S404 where the controlling unit 28 judges whether the image taking period has elapsed.

On the contrary, if a scan skipping request has been received (step S408: Yes), the controlling unit 28 judges whether the current bed position is the last bed position (step S409). Also, if the image taking period “Tb” has elapsed without receiving any scan skipping request (step S404: Yes), the controlling unit 28 judges whether the current bed position is the last bed position (step S409).

In this situation, if the current bed position is not the last bed position (step S409: No), the driving unit 13 moves the next bed position of the subject P into the FOV under the control of the controlling unit 28 (step S410), and the process returns to step S403 where the controlling unit 28 causes an image taking process to start at the post-move bed position.

On the contrary, if the current bed position is the last bed position (step S409: Yes), the controlling unit 28 ends the image taking process (step S411), and the process is thus ended.

As explained above, according to the fourth embodiment, even if it is determined that it is possible to reconstruct the PET image data that is suitable for the diagnosis purpose on the basis of the objective criterion based on the quantity of pair annihilation events accumulated during the image taking process, the operator is able to judge whether the scan skipping process should be performed or not by viewing the actual reconstructed image and checking, once again, whether the reconstructed image has a sufficient level of image quality. As a result, according to the fourth embodiment, for example, it is possible to reduce the possibility of having to perform the image taking process again and to thus improve the workflow of the PET examination.

### Fifth Embodiment

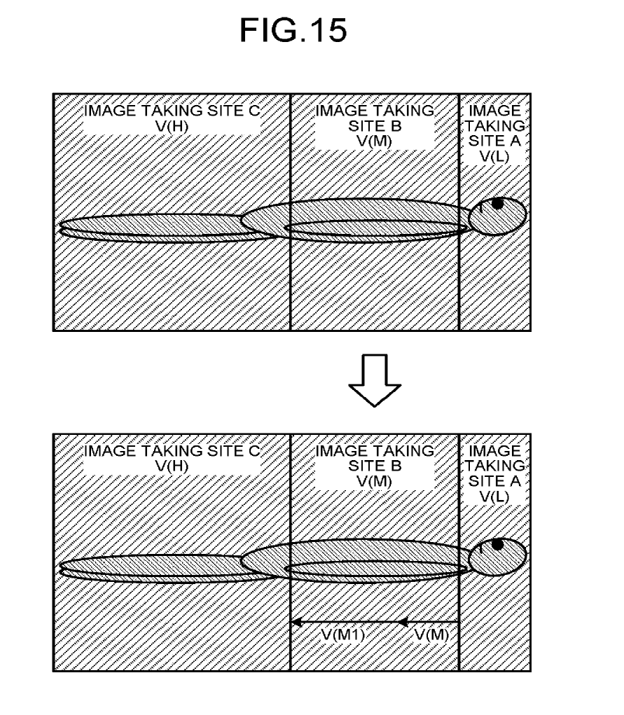

In the first to the fourth embodiments described above, the controlling unit 28 performs the scan skipping process by controlling the driving unit 13, in the examples where the image taking condition is changed with respect to the image taking process that is performed after it is determined that the data that has already been acquired from the image taking site currently being imaged by using the step-and-shoot method satisfies the predetermined condition. In a fifth embodiment, an example will be explained with reference to FIG. 15, in which the controlling unit 28 changes the image taking condition of the image taking process that is performed after the determination, by performing a control process that is different from the scan skipping process. FIG. 15 is a drawing for explaining the fifth embodiment.

In the fifth embodiment, the driving unit 13 is able to change the moving speed of the subject P to an arbitrary speed, under the control of the controlling unit 28. In the fifth embodiment, the operator makes an image taking plan, by making use of the mechanism of the driving unit 13 that is able to change the moving speed to an arbitrary speed.

For example, as illustrated in the top section of FIG. 15, for image taking site A used for imaging the head/neck part, the operator sets a “moving speed: V(L)” by which the subject P is moved at a low speed, in order to acquire as much count information as possible and to obtain image data having high resolution. In another example, as illustrated in the top section of FIG. 15, for image taking site B used for imaging the chest and the abdomen, the operator sets a “moving speed: V(M)” by which the subject P is moved at a speed slightly lower than “V(L)”, in order to obtain image data having high S/N ratio. In yet another example, as illustrated in the top section of FIG. 15, for image taking site C used for imaging the lower limbs, the operator sets a “moving speed: V(H)” by which the subject P is moved at a low speed, because it is sufficient if image data having a certain level of S/P ratio is obtained. In addition, for example, the operator makes a setting that the image taking processes are to be performed at image taking site A, image taking site B, and image taking site C, in the stated order.

Subsequently, after the image taking process at image taking site A is started, the controlling unit 28 judges whether the data that has already been acquired from the image taking site currently being imaged satisfies the predetermined condition, by performing any of the processes explained in the first to the fourth embodiments. In this situation, for example let us discuss an example in which the controlling unit 28 has determined that the data that has already been acquired from image taking site B currently being imaged at the “moving speed: V(M)” satisfies the predetermined condition. In that situation, because the image quality of the image data is guaranteed even at the “moving speed: V(M)”, it is possible to make the moving speed higher in order to improve the workflow. For this reason, as illustrated in the bottom section of FIG. 15, for example, the controlling unit 28 controls the driving unit 13 so as to change the moving speed to V(M1) that is higher than V(M).

When the controlling unit 28 has determined that the data that has already been acquired from image taking site B satisfies the predetermined condition under the image taking condition of V(M1), the controlling unit 28 may change the moving speed to a speed that is higher than V(M1). Further, when the controlling unit 28 has determined that the data that has already been acquired from image taking site B no longer satisfies the predetermined condition under the image taking condition of V(M1), the controlling unit 28 may change the moving speed back to V(M).

As a result of the control described above, in the fifth embodiment, it is possible to adjust the image taking period by changing the moving speed, while guaranteeing that it is possible to obtain the image data having the level of image quality desired by the operator by performing the judging process based on the predetermined condition. As a result, according to the fifth embodiment, it is possible to, for example, shorten the image taking period required by the entire-body image taking process set by the operator. It is therefore possible to improve the workflow of the PET examination.

The controlling method described in any of the first to the fifth embodiments is also applicable to, besides a PET apparatus, a Positron Emission computed Tomography/Computed Tomography (PET-CT) apparatus in which a PET apparatus is integrated together with an X-ray CT apparatus or a Positron Emission computed Tomography/Magnetic Resonance Imaging (PET-MRI) apparatus in which a PET apparatus is integrated together with an MRI apparatus.

Further, the controlling method described in any of the first to the fifth embodiments is applicable to a Single Photon Emission computed Tomography (SPET) apparatus that reconstructs Single Photon Computed Tomography (SPCT) image data by using the count information of gamma rays emitted due to decay events of a radioisotope that is specifically taken into tissues in the body of the subject P. Further, the controlling method described in any of the first to the fifth embodiments is also applicable to a SPET-CT apparatus in which a SPET apparatus is integrated together with an X-ray CT apparatus or a SPET-MRI apparatus in which a SPET apparatus is integrated together with an MRI apparatus.

Further, the constituent elements of the apparatuses that are illustrated in the drawings in the first to the fifth embodiments are based on functional concepts. Thus, it is not necessary to physically configure the elements as indicated in the drawings. In other words, the specific modes of distribution and integration of the apparatuses are not limited to the ones illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a Central Processing Unit (CP) and a computer program that is analyzed and executed by the CPU or may be realized as hardware using wired logic.

Furthermore, the controlling methods explained in the first to the fifth embodiments may be realized by causing a computer such as a personal computer or a workstation to execute a controlling computer program (hereinafter, a “controlling program”) that is prepared in advance. The controlling program may be distributed via a network such as the Internet. Further, it is also possible to record the controlling program onto a computer-readable recording medium such as a hard disk, a flexible disk (FD), a Compact Disk Read-Only Memory (CD-ROM), a Magneto-optical (MO) disk, a Digital Versatile Disk (DVD), or the like, so that a computer is able to read and execute the controlling program from the recording medium.

According to at least one aspect of the exemplary embodiments described above, it is possible to improve the efficiency of the medical examination.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A nuclear medical imaging apparatus comprising:
a driving unit that moves each of a plurality of image taking sites of a subject into an image taking region; and
a controlling unit that, when an image taking process for the subject is performed at each of the plurality of image taking sites, if data that has already been acquired from one of the image taking sites currently being imaged is determined to satisfy a predetermined condition, changes an image taking condition of the image taking process that is performed after the determination.

2. The nuclear medical imaging apparatus according to claim 1, further comprising: an acquiring unit that acquires data related to decay events of a radioisotope, wherein
if the data that has already been acquired by the acquiring unit from one of the image taking sites currently being imaged is determined to satisfy the predetermined condition, the controlling unit controls the driving unit so that the image taking process is started at a next image taking site following the image taking site currently being imaged.

3. The nuclear medical imaging apparatus according to claim 2, further comprising: an image reconstructing unit that reconstructs image data on a basis of count information of the decay events, wherein
the controlling unit causes the image reconstructing unit to reconstruct the image data on a basis of the count information of the decay events that have already been acquired from the one of the image taking sites currently being imaged and causes a display unit to display the image data, and if an imaging ending instruction with respect to the image taking site is received via an input unit from an operator who views the image data displayed on the display unit, the controlling unit determines that the predetermined condition is satisfied.

4. The nuclear medical imaging apparatus according to claim 2, wherein if a count of the decay events that have already been acquired from the one of the image taking sites currently being imaged has exceeded a predetermined threshold value, the controlling unit determines that the predetermined condition is satisfied.

5. The nuclear medical imaging apparatus according to claim 4, wherein if the count of the decay events that have already been acquired from the one of the image taking sites currently being imaged has exceeded the predetermined threshold value, the controlling unit inquires an operator whether the image taking process at the image taking site should be ended or not, and if an imaging ending instruction is received from the operator via an input unit, the controlling unit determines that the predetermined condition is satisfied.

6. The nuclear medical imaging apparatus according to claim 4, wherein the controlling unit judges whether the predetermined condition is satisfied or not, by using a threshold value that is set for each of the plurality of image taking sites.

7. The nuclear medical imaging apparatus according to claim 4, wherein the controlling unit judges whether the predetermined condition is satisfied or not, by using either a threshold value that is set in accordance with at least one of body information and pathological information of the subject or a threshold value that is set for each of the plurality of image taking sites in accordance with at least one of body information and pathological information of the subject.

8. The nuclear medical imaging apparatus according to claim 3, wherein, if a count of the decay events that have already been acquired has not exceeded a threshold value at a point in time when the imaging ending instruction with respect to the image taking site currently being imaged is received from the operator via the input unit, the controlling unit inquires the operator whether the image taking process at the image taking site should be ended or not, and if an imaging ending instruction is received again from the operator via the input unit, the controlling unit determines that the predetermined condition is satisfied.

9. The nuclear medical imaging apparatus according to claim 8, wherein the controlling unit makes the inquiry to the operator by using the threshold value that is set for each of the plurality of image taking sites.

10. The nuclear medical imaging apparatus according to claim 8, wherein the controlling unit makes the inquiry to the operator by using either the threshold value that is set in accordance with at least one of body information and pathological information of the subject or the threshold value that is set for each of the plurality of image taking sites in accordance with at least one of body information and pathological information of the subject.

11. The nuclear medical imaging apparatus according to claim 4 further comprising: an image reconstructing unit that reconstructs image data on a basis of count information of the decay events, wherein
if the count of the decay events that have already been acquired from the one of the image taking sites currently being imaged has exceeded the predetermined threshold value, the controlling unit causes the image reconstructing unit to reconstruct the image data on a basis of a counted result of the decay events that have already been acquired, causes a display unit to display the image data, and if an imaging ending instruction with respect to the image taking site is received via an input unit from an operator who views the image data displayed on the display unit, the controlling unit determines that the predetermined condition is satisfied.

12. The nuclear medical imaging apparatus according to claim 2, wherein the acquiring unit acquires data related to pair annihilation events as the data related to the decay events.

13. A controlling method comprising:
a process performed by a driving unit to move each of a plurality of image taking sites of a subject into an image taking region; and
a process performed by a controlling unit to, when an image taking process for the subject is performed at each of the plurality of image taking sites, if data that has already been acquired from one of the image taking sites currently being imaged is determined to satisfy a predetermined condition, change an image taking condition of the image taking process that is performed after the determination.
